(19) United States
(12) Patent Application Publication
(10) Pub. No.: US 2019/0390269 A1
(43) Pub. Date: Dec. 26, 2019

(54) METHOD FOR DETECTING KNOWN NUCLEOTIDE MODIFICATIONS IN AN RNA

(71) Applicant: Johannes Gutenberg-Universitaet Mainz, Mainz (DE)

(72) Inventors: Mark HELM, Mainz (DE); Ralf HAUENSCHILD, Mainz (DE); Lyudmil TSEROVSKI, Mainz (DE); Stephan WERNER, Mainz (DE); Andreas HILDEBRANDT, Saarbruecken (DE); Jennifer LECLAIRE, Mainz (DE); Thomas KEMMER, Mainz (DE)

(73) Assignee: Johannes Gutenberg-Universitaet Mainz, Mainz (DE)

(21) Appl. No.: 16/483,896

(22) PCT Filed: Feb. 21, 2018
(86) PCT No.: PCT/DE2018/000044

§ 371 Date: Aug. 6, 2019

(30) Foreign Application Priority Data
Mar. 4, 2017 (DE) 10 2017 002 092.2

(51) Int. Cl. C12Q 1/6869; G16B 30/00

(52) U.S. Cl. CPC C12Q 1/6869; G16B 30/00; C12Y 207/07049; C12Q 2525/117; C12Q 2521/107

(57) ABSTRACT

The method comprises: the reverse transcription of the template RNA, the amplification and high-throughput sequencing of the cDNAs obtained in this way, the mapping of the sequenced cDNAs/reads to the reference genome using computerized alignment methods, a computerized evaluation of the mapping results with regard to the reverse transcription event pattern (the RT signature) at the nucleotide positions and feeding the digitalised data of the RT signatures into a computerized machine learning based classification system. Reverse transcription is carried out in parallel reaction batches with different reverse transcriptases and/or under different reaction conditions. The evaluation of the mapping results with regard to the RT signature is carried out using the events ‘arrest’ and/or ‘readthrough with mismatch’ and/or ‘readthrough with sequence gap(s)’. RT signature data obtained using the parallel reaction batches are fed into the classification system.

The present invention relates to a method for detection, i.e. determination of number and position (locus), of a selected known nucleotide modification in one or more RNAs (incl. Transcriptome).

The abbreviations used in the context of this description of the invention are defined as follows:

RT=reverse transcription
RTase=reverse transcriptase
RT signature=reverse transcription signature
RNA=ribonucleic acid
mRNA=messenger RNA
tRNA=transfer RNA
rRNA=ribosomal RNA
NGS=Next-Generation-Sequencing=high-throughput sequencing
m1A=N1-methyladenosine (m¹A)
m1G=N1-methylguanosine (m¹G)
m2,2G=N2,N2-dimethylguanosine (m²₂G)
PCR=polymerase chain reaction
dNTP=deoxynucleotide triphosphate

The transcriptome, —meaning the entirety of the RNA transcripts (i.e. all genes read or transcribed by the RNA polymerase) of a genome of a cell or of a cell type or of an organism, in particular mRNAs, tRNAs and rRNAs, but also others non-coding RNAs—, plays a crucial role in various aspects of gene expression, cell development and cell function. Errors in the transcriptome, for example due to modified nucleotides in an mRNA or tRNA or rRNA, can lead to diseases. The identification and characterization of various types of modifications of RNA bases in different types of RNA have become increasingly important in recent years. Interest in current research is growing, and this field gains more and more importance.

The methods known in the art for the detection of modified (altered) nucleotides in RNAs are based on their reverse transcription (=translation) into cDNA by means of reverse transcriptases (abbreviated: RTases) and the subsequent sequence analysis of these cDNAs, i.e. sequencing and mapping (=assignment) to a known reference genome or reference transcriptome, respectively.

Thereby, the cDNAs obtained with a specific RTase during reverse transcription (abbr.: RT) of a RNA selected as template (synonyms: matrix, template) are first amplified and then sequenced.

The resulting sequencing data of the cDNAs, the so-called “reads”, are compared with the genomic reference sequence, and in the course of the so-called “mapping” the sequenced cDNAs/reads are assigned to the reference genome or reference transcriptome.

A special and specific transcriptional behavior of reverse transcriptase (RTase) in the transcription of RNA into cDNA at the sites of a nucleotide modification serves as a starting point for the detection of modified nucleotides.

This special and specific reverse transcription behavior is manifested by the fact that multiple behavioral variants and, as a result, various special (i.e. aberrant from correct reverse transcription) reverse transcription events may occur at the site of nucleotide modification.

These include in the prior art, in particular, (1.) the blockage (arrest) of the reverse transcription, resulting in a correct but incomplete so-called arrest product, and (2.) the incorporation of a false (naturally non-corresponding) dNTP at the position of the modified RNA nucleotide into the cDNA resulting in a complete but incorrect translation product, a so-called ‘mismatch read-through product’.

The type and number of different RT events form a characteristic event pattern, the so-called reverse transcription signature (in the following: RT signature) at each individual nucleotide position.

In the prior art, the RT signature for the nucleotide positions of a subject RNA (the template RNA) is principally characterized by the characteristics of arrest events and mismatch read-through events (i.e. Read-through events with misincorporated or mismatched cDNA building blocks).

For the detection of a particular (presumably) existing nucleotide modification in a template RNA, e.g. the N1-methylation of adenosine to N1-methyladenosine (m1A), the mapping results obtained after reverse transcription, amplification, sequencing and mapping for this template RNA, are examined and evaluated in order to determine whether and if so at which nucleotide position which RT events occur in what frequency, and thus what the RT signatures look like for each nucleotide position.

From the RT signature obtained for the template RNA in question, it is possible to deduce existing nucleotide modifications. If a particular characteristic RT signature could be determined for a specific nucleotide modification, as has been done in the prior art for m1A, the presence of that relevant nucleotide modification in the template can be concluded by comparison with this known and modification-specific RT signature.

The template RNA may be a particular RNA species, as well as a group of different RNA species.

Amplification and sequencing of the cDNAs are usually carried out in the prior art with sequencing methods based on high throughput methods in the form of massive parallel sequencing, the so-called “Next-Generation Sequencing” (NGS), wherein the acquired sequence data are output in digital form.

A known next-generation sequencing (NGS) method is the so-called “bridge amplification sequencing”. Here, a different adapter DNA sequence is introduced at each end of the (double-stranded) DNA to be sequenced. Subsequently, the DNA is denatured, after dilution (single-stranded) hybridized to a carrier plate and amplified by bridge amplification. As a result, individual regions (clusters) of amplified DNA are formed on the carrier plate, with the DNAs within a cluster having the same sequence. In a sequencing-by-synthesis-related PCR reaction (i.e. a PCR reaction where sequencing is done during synthesis), modified nucleotides coupled to a reversible 3′ blocker and a fluorescent label (each of the four nucleotides coupled with a different coloured fluorescent label) are used, which force the polymerase to incorporate only one nucleotide per cycle. The built-in nucleotide per cycle in a cluster is detected.

The mapping is preferably carried out by means of computerized alignment methods familiar in the prior art, and also the evaluation (analysis) of the mapping results with regard to the reverse transcription event pattern (the RT signature) is usually computerized.

The characteristic RT signature described in the prior art for m1A was determined using computerized, automatic and supervised machine learning-based classification techniques known and common in the art.

For the nucleotide modification m1A in tRNA and rRNA as template RNAs, Hauenschild et al. (Nucleid Acid Research, 2015) describe the simultaneous analysis of the RT signature features arrest and mismatch read-through by using the NGS sequencing method RNA-Seq.

Applying this NGS methodology to a variety of native and synthetic RNA preparations as template RNAs and by further bioinformatic processing of the generated data, including classification using computerized and machine learning-based classification techniques, the authors found that for m1A modifications (i.e. at m1A modification sites or at nucleotide positions with m1A modification) a characteristic reverse transcription event pattern, i.e. an RT signature characteristic of this nucleotide modification site, exhibiting transcriptional arrest products and transcription read-through products as significant feature components.

The determined RT signature for m1A (i.e., at m1A sites) was used for the verification and confirmation of suspected objects. Putative positions of m1A in the sequences of several human RNAs could be confirmed, and in tRNA of Trypanosoma brucei previously unknown m1A positions were detected by signature comparison and sequence homology.

Thus, Hauenschild et al. (2015) demonstrated that the RT signature of a reverse transcriptase (RTase) at a m1A site consists of arrest rates and mismatch rates, which can be used to identify, characterize, and localize m1A sites in tRNA and rRNA.

Other publications have already demonstrated approaches for the recognition of modification sites at a transcriptome-wide level. Dominissini et al. (2016) describe the strategies of antibody-based, methylated RNA immunoprecipitation sequencing (MeRIP-Seq) for the detection of the modification m1A. Ni-methyladenosine-containing RNA fragments were enriched using anti-m1A antibodies, and by coupling with a chemical method, attempts were made to localize the m1A-modifications. This procedure allows identification and/or prediction of m1A sites/positions in a single-nucleotide resolution only in exceptional cases and with limited reliability. Linder et al. (2015) describe a mapping of the modification N-6-methyladenosine (m6A) in human and mouse mRNA with the miCLIP method (miCLIP=methylation individual-nucleotide-resolution crosslinking and immunoprecipitation) using ultraviolet light-induced antibody RNA crosslinking and reverse transcription. However, significant amounts of false-negative and false-positive results are obtained, and as a result, the predictive power for actual modification sites is tightly limited.

It is an object of the present invention to obviate or at least mitigate these disadvantages of the prior art, in particular to increase the known methodology for detecting positions of modified nucleotides in RNAs with regard to detection accuracy and predictive quality, and to further develop them for applications for the analysis of RNA modifications other than the m1A modification, in view of the goal of transcriptome-wide mapping of RNA modifications.

A solution to this problem is to provide a method for determining number and position (locus) of a selected (predetermined) known nucleotide modification in one RNA or multiple RNAs (incl. Transcriptome), the so-called template RNA(s), comprising the following steps in the specified order:

(1) Reverse transcription of the template RNA(s) using the enzyme reverse transcriptase (“RTase”) and creating a cDNA library containing the reverse transcription products (=cDNAs) of the reverse transcriptase used with this/these template RNA(s),
(2) Amplifying the cDNAs and sequencing the amplified cDNAs using a high-throughput sequencing method (Next-Generation-Sequencing (NGS)-Method), the recovered sequence data being output in digital form, i.e. in the form of reads,
(3) Adapter trimming (=removal of the adapter sequences) and mapping (=assignment) of the sequenced cDNAs/reads to the reference genome or reference transcriptome by means of computerized alignment methods,
(4) computerized evaluation (analysis) of the mapping result with respect to the reverse transcription event pattern, the RT signature, using the events ‘arrest’ and/or ‘read-through with mismatch’ as RT signature feature(s), and diagnosing the RT signature at each nucleotide position of the template RNA(s),
(5) Feeding the (digitized) data (sets) of RT event patterns/RT signatures into a computerized, automated, machine-learning based classification system,
wherein in a first phase (I) of the method, the calibration phase, steps (1) to (5) are carried out with one or several different RNAs as template RNAs, wherein this/these RNA(s) are known and identified and annotated with respect to nucleotide sequence and optionally present nucleotide modification(s), and RT signatures determined in step (5) of nucleotide positions with the known nucleotide modification and of nucleotide positions of the same nucleoside without nucleotide modification are fed into the classification system, and during training and self-testing (classification) runs the classification system implicitly creates and optimizes (“learns”) the (characteristic) profile of the RT signature (i.e. the characteristic quantitative expression of the RT signature features) at the nucleotide position having the nucleotide modification, and (consequently) as a classification result, it determines and indicates those positions on the (each) template RNA(s) which have an RT signature that approximately or fully matches this (characteristic) profile, i.e. that is similar to or identical to this profile, and thus indicates the presence of the relevant nucleotide modification at these positions,
and wherein in a second phase (II) of the method, the application or examination phase, steps (1) to (5) are carried out with one or more unknown test RNA(s) to be examined, as template RNA (s), and steps (1) to (4) are carried out under the same conditions as in Phase (I), and RT signatures determined in step (5) of (preferably all or nearly all) nucleotide positions of the test template RNA(s) are fed into the classification system, and on the basis of the (characteristic) profile implicitly learned in phase (I) step (5) the classification system classifies the (and preferably each of the) entered RT signatures with regard to the criterion to what extent (i.e. to what rate or degree) they are similar to or match this profile, and wherein classification results with the statement “similar” or “approximately matching” or “matching” (i.e. classification results corresponding to the statement “similar” or “approximately matching” or “matching” indicate the presence of the subject nucleotide modification in the test template RNA(s) at the nucleotide position with this RT signature.

According to the invention this method is characterized in that:
in step (1) of phase (I) and phase (II) of the method, the reverse transcription of the template RNAs is carried out in two or more reaction mixtures and reaction runs with different RTases under the same reaction conditions and/or with the same RTase(s) under different reaction conditions per batch, wherein a cDNA library is obtained with/from each batch,
and that in step (4) of phase (I) and phase (II) of the method, for evaluating the mapping results with respect to the RT signature, the event (s) ‘arrest’ and/or ‘read-through with mismatch’ and/or the additional event ‘read-through with sequence gap(s) (jump/jumps)’ are determined and evaluated as RT signature feature(s),
and that in step (5) of phase (I) and phase (II) of the method data (sets) of (all or almost all) RT signatures (or at least of RT signatures determined at nucleotide positions with the base type of the respective nucleotide modification) from the cDNA libraries obtained in step (1), which were obtained with the different RTases under the same reaction conditions and/or with the same RTase(s) under different reaction conditions, are fed into the classification system.

With other words:

The inventive method for determining number and position (locus) of a selected (predetermined), known nucleotide modification in one or more to RNAs (incl. Transcriptome) be examined, the template RNA(s), consists of two phases (I) and (II):

In Phase I, the calibration phase, the following steps are performed in the specified order:

(1) Reverse transcription of one or several different known RNA(s), identified and annotated with respect to their nucleotide sequence and to the eventually existing selected nucleotide modifications, as template RNA(s) (preferred are synthetic RNAs or RNAs isolated from natural sources according to database information i.e. from MODOMICS according to Machnicka et al., 2013), in two or more parallel reaction mixtures and reaction runs with different RTases (including those modified by mutations specifically for this purpose) under the same reaction conditions, and/or with the same RTase(s) under different reaction conditions per batch, and creating cDNA libraries, one each per reaction run, wherein each created cDNA library contains the reverse transcription products (cDNAs) of the RTase used in the respective reaction run from the template RNA(s) used therein.
(2) For each cDNA library (obtained in step 1) amplification of the cDNAs and sequencing of the amplified cDNAs are performed by a high-throughput sequencing method. The sequence data obtained, i.e. the sequence information obtained of the individual cDNAs (synonym: reads), are output in digital form. A “sequencing with bridge amplification”, e.g. the Illumina sequencing procedure, is preferred here.
(3) Adapter trimming (=removal of adapter sequences) and mapping (=assignment) of the sequenced cDNAs or reads to the reference genome or reference transcriptome using computerized alignment techniques. The use of a computer-based method for sequence alignment and sequence analysis, z. B. the Bowtie 2 software is preferred here.
(4) Computer-aided evaluation (computational analysis) of the mapping result(s) with respect to the reverse transcription event pattern, the so-called RT signature, using the events ‘arrest’ and/or ‘read-through with mismatch’ and/or ‘read-through with sequence gap(s) (synonym: jump(s))’ as defining/characteristic feature(s), and diagnosis of the RT signature (i.e. the transcription event pattern) at preferably each nucleotide position of the template RNA(s).
(5) Feeding the (digitized) data (data sets) of the RT signatures—of all or almost all or at least of those RT-signatures determined at the nucleotide positions with the base type of the respective nucleotide position—into a computer-based, automatic classification system (synonyms: classification method, classifier) based on machine and supervised learning, e.g. into a Random Forest classifier, and training this (learning) classification system on the particular (characteristic, typical) profile of the RT signatures obtained in step (4) (i.e. on the characteristic quantitative expression of the RT signature features) for or at the nucleotide position(s) with the respective nucleotide modification (i.e. the nucleotide position(s) having the relevant nucleotide modification) in such a way, that it determines and indicates as classification result those positions on the (each) template RNA(s), which have an RT signature that approximately or completely match this profile, i.e. which are similar or coincident/consistent with it, and thus indicate the presence of the relevant nucleotide modification at these positions.

In Phase II, the analysis or examination phase with at least one test RNA, the following steps are carried out in the specified order:

(1) Reverse transcription of the test RNA(s) to be tested as template RNA(s) under the same conditions as in phase I step (1), i.e. with the RTase(s) and reaction conditions used in phase I step (1), and creation of cDNA libraries (one per batch) comprising the reverse transcription products of the particular RTase(s) used for this test template RNA(s).
(2) Amplifying the cDNAs obtained in step (1) and sequencing the amplified cDNAs using the method used in step I step (2), wherein the sequence data (reads) obtained are output in digital form.
(3) Assignment (=mapping) of the sequenced cDNAs/reads to the reference genome or reference transcriptome by means of the computer-aided alignment method used in phase I step (3).
(4) computer-aided evaluation (analysis) of the mapping result analogous to phase I step (4) with respect to the RT signatures (i.e. the reverse transcription event pattern) using the events ‘arrest’ and/or ‘read-through with mismatch’ (and here absolute rate and/or individual rates of the different mismatch compositions (mismatches)) and/or ‘read-through with sequence gap(s) or Jump(s)’ (and here absolute rate=total jump rate and/or individual rates of the various gap or jump variants) as defining/characteristic feature(s).
(5) Feeding the (digitized) data or data sets of the determined RT signatures into the computer-based classification system from phase I step (5) based on monitored machine learning and trained on the special profile for the nucleotide modification concerned, in such a manner that a classification is made for each entered RT signature to what extent it is similar to or coincide with this profile. (This means that each RT signature entered is classified according to the criterion of how much or to what extent or degree it resembles or matches this profile). Classification results corresponding to the statement “similar” or “approximately matching” or “matching” indicate the presence of the subject nucleotide modification in the test template RNA(s) at the nucleotide position with this RT Signature.

The core result of the classification is the indication of the identified positions on the test template RNA(s) that have an RT signature that approximately or fully matches this (specific) profile, i.e. that is similar or consistent/coincident with it, and thus indicates the presence of the nucleotide modification concerned at those positions.

Preferably, for each of these determined and displayed positions, a numerical score is given on a one-dimensional numeric rating scale as a measure of the quality of the match.

The method according to the invention is based on the surprising findings:

(i) The RT signature at a nucleotide modification site depends not only on the type of nucleotide modification, but also on the RTase type (the RTase species). Because of their very specific and characteristic behavior at the site of a nucleotide modification, an RTase type-specific RT signature is obtained at this nucleotide position.
(ii) By combining at least two RTases of different types in reverse transcription, surprisingly strong performance improvements are obtained in prediction by classifiers. Two or more (parallel) reaction mixtures and reaction runs with mutually different RTases under the same reaction conditions and/or with the same RTase(s) under different reaction conditions per batch lead to a significantly improved accuracy of the prediction (classification) of whether or not the relevant (sought) nucleotide modification is present at a particular nucleotide position. Comparative experiments with on the one hand (a) two parallel RT batches using two different RTases and on the other hand (b) batches using only a single RTase species, each have shown that the accuracy of the prediction (classification) in case (a) was significantly greater than in case (b), indicating that in case (a) there is a synergistic effect.
(iii) The RT signature is characterized not only by the special features (special RT events) arrest and mismatch read-through (broken down into overall rate and single rates of the various mismatches), but also by the feature “read-through events with sequence gap(s) (synonyms: jump(s))” abbr. “jump-read-through”, i.e. by events in which the RTase skips the site of nucleotide modification. This feature “jump-read-through” can (also) be further broken down into: total jump rate, rate of direct single jumps, rate of delayed single jumps and rate of double jumps.

In the course of the investigations on which this invention is based, it was surprisingly found that the reverse transcription event of RTases at a nucleotide modification site can be not only a transcription termination or read-through with mismatch, but also a jump of the respective RTase across the position of the modified nucleotide, resulting in characteristic gaps in the sequence reading. Such jumps have been found especially with RTases revealing a high coverage, i.e. having a strong read-through capacity. Single and double jumps can be distinguished, and the single jumps can be either direct or delayed single jumps, that is, the (skipped) gap is either directly at the m1A site or at the location of its 5′ adjacent neighbor, known as −1 position. Double jumps lead to and appear as gaps at both positions m1A- and −1.

According to a preferred embodiment of the method according to the invention, in step (4) of phase (I) and phase (II) of the method, for the evaluation of the mapping results with regard to the RT signature, all three events ‘arrest’ and ‘read-through with mismatch’ and ‘read-through with sequence gap(s) (jump(s))’ are determined qualitatively and quantitatively and evaluated as defining/characteristic features. This may enhance the conciseness and uniqueness of each RT signature.

According to a likewise preferred embodiment, in phase (I) step (1) of the method, the analogous reaction mixtures and reaction runs are carried out with at least two reverse transcriptases (“RTases”), whose RT signatures at or for the nucleotide modification site in question have a different pattern with regard to the weighting (synonym: importance) of their RT signature features. Preferably, the patterns differ in the weighting of at least one of the features in such a way that this feature is pronounced in the RT signature of the one RTase and weak or at least significantly less pronounced in the RT signature of the other RTase.

Particularly preferred different patterns are those which have at least two RT signature features (M1 and M2, e.g. the arrest rate and the mismatch rate) that show a reciprocally opposite pattern. That is, of the features in question, e.g. M1 and M2, in one RTase (A) feature M1 is strongly and feature M2 is only weakly pronounced, while in the other RTase (B) the ratio is reversed, namely M1 is weakly and M2 is strongly pronounced.

According to the invention, the RTases used in the step (1) of calibration phase (Phase I) and application or investigation phase (phase II) may well be those which were generated for this purpose by mutations.

According to the invention, the different reaction conditions in step (1) of phase I and phase II, may be in particular (a) different concentrations of dNTPs, and/or (b) different divalent cations, in particular Mg2+ and Mn2+, and/or (c) different concentrations of divalent cations and/or (d) different pH values and/or (e) different temperatures and/or (f) different concentrations of polyethylene glycol (PEG).

The method according to the invention has already proven itself in practice in the analysis of the RNA modification m1A. For the detection of other RNA modifications, especially those for which the sequencing data analysis provides a typical profile of the RT signature, as e.g. guanosine derivatives N1-Methyl guanosine (m1G) and N2,N2-dimethylguanosine (m2,2G), it is also suitable and intended for. An embodiment of the method according to the invention is therefore in particular that the nucleotide modification is a nucleoside methylation, in particular a N1-methylation of adenosine or guanosine.

As a high-throughput sequencing method (NGS method) in step (2) of phase I and phase II of the method according to the invention, a sequencing with bridge amplification, in particular an illumina sequencing method, has proven to be well suited in practice.

For the mapping, i.e. the assignment of the sequenced cDNAs/reads to the reference genome or reference transcriptome by computer-aided alignment methods in step (3) of Phase I and Phase II of the method according to the invention, a computer-based method for sequence alignment and sequence analysis, such as e.g. the Bowtie 2 software, has proved to be well suited in practice.

As computer-based automated machine learning-based classification system in step (5) of phase I and phase II of the method of the invention, a Random Forest classifier has been found to be well suited in practice.

In a preferred embodiment of the method according to the invention, for carrying out steps (3) to (5) the sequence data obtained in step (2) are fed into a bioinformatics pipeline which controls the combination of steps (3) to (5). According to the invention, such a bioinformatics pipeline, i.e. the software program that combines or links the operations (3) to (5) in the prescribed order, can be created with the programming language Python (Version v2.7.6).

According to the invention the known RNAs used in the calibration phase (phase I) step (1) are preferably synthetic RNAs of known sequence including known positions of the relevant (selected) nucleotide modification, or natural RNAs isolated on the basis of database information, the sequence of which, including the positions of the relevant (selected) nucleotide modification, is well understood according to the relevant database entries.

In a preferred embodiment of the method according to the invention, in step (5) of phase II and optionally also of phase I, for each classification result a numerical score is given on a one-dimensional numerical rating scale as a measure of the quality of the match.

The present invention also provides a kit for carrying out the method according to the invention. This kit comprises at least two RTases whose RT signatures at the relevant nucleotide modification site, with respect to the weighting of the RT signature features (arrest rate, overall mismatch rate, single mismatch rates of the respective mismatched nucleotides, total jump rate, rate of direct jumps, rate of delayed jumps, double jumping rate), have a different, preferably opposite pattern in at least one of the RT signature features. Opposing pattern in at least one RT signature feature here means that e.g. the feature M1 is strongly pronounced in RTase A and only weak in RTase B. Alternatively or additionally, the kit comprises at least two different premixed reaction batches (synonyms: reaction mixtures, buffer mixtures) which preferably differ in the concentration of dNTPs and/or od divalent cations and/or of polyethylene glycol (PEG) and/or in the nature of the divalent cations (especially Mg2+ and Mn2+) contained and/or in the pH-value. For carrying out the method according to the invention with such a kit, additionally only the template RNA(s) is(are) required.

The method of the invention is a powerful tool for the detection of modified nucleotides in RNA on the basis of the RT signature at the site of modification, i.e. on the basis of the analysis of the modification-specific behaviour of RTase during the reverse transcription of the RNA to cDNA. It allows for accurate localization of RNA modifications in a single nucleotide resolution, and thus, for example, a much more accurate identification and prediction of m1A sites compared with conventional methods, and in principle it is equally well suited for analysis of other modifications, such as e.g. m1G or m2,2G.

Carrying out the reverse transcription of the template RNA(s) in step (1) of phase (I) and phase (II) of the method in two or more parallel (analogous) reaction batches and reaction runs with RTases different from each other and/or under different reaction conditions per batch, and the comparison of the RT signatures thus obtained and usually not completely identical for the same nucleotide modification site, enables to clarify the characteristic features of the RT signature for the relevant nucleotide modification site more accurate and further specify. The more succinctly and more specifically the characteristic features for the RT signature can be given at a particular nucleotide modification site, the more accurate it can be determined for the RT signature of the reverse transcription of a test template RNA (e.g. from a patient sample), whether or not it represents an embodiment of this known RT signature, i.e. whether or not the nucleotide modification in question is present in the test template RNA(s).

The method according to the invention is a universal method for the transcriptome-wide detection of RNA modifications involving very specific properties of the reverse transcription or the RTase, respectively, which enables detection of individual, modified nucleotides within the sequence solely on the basis of their characteristic RT signature. An accumulation obtained by immunoprecipitation of sequence regions which (presumably) contain the nucleotide modification can be completely omitted/dispensed with here.

The method according to the invention can be applied and targeted in the field of clinical diagnostics by analytical service providers or medical-diagnostic laboratories for further developing personalized medicine with regard to patient-specific diagnostics. Especially in view of the rapidly growing interest in the effects and functions of RNA modifications, many new insights in this field of activity can be expected in the coming years, making the precise determination of modified sequence positions or nucleotide positions all the more important.

Because of its performance in locating the exact position of the modifications and minimizing false results, the method of the invention enables to make serious statements about their effect and function and to make routine application in an economical manner. Analytical service providers or clinical diagnostic laboratories can analyse patient-derived RNA samples and can generate a report of classified modification candidates, providing additional information for the patient's diagnostic work-up.

The invention is explained in more detail on the basis of the following exemplary embodiments and the figures and tables mentioned therein.

It is shown in:

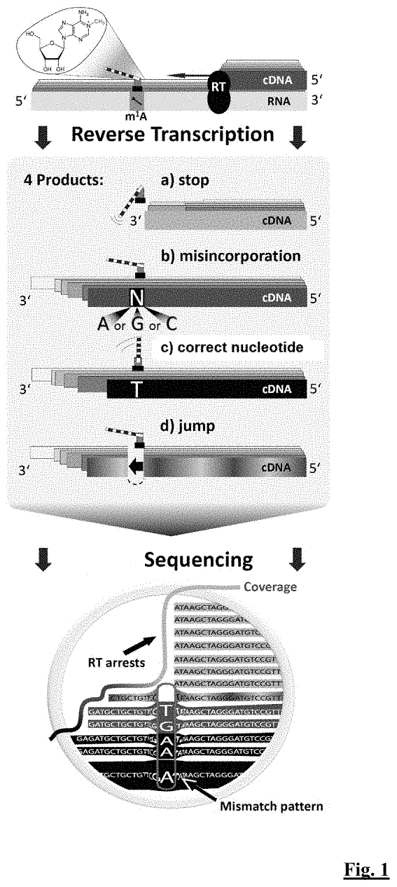

FIG. 1: The inventive principle of generation and analysis (evaluation) of RNA sequencing data for the detection of m1A residues.

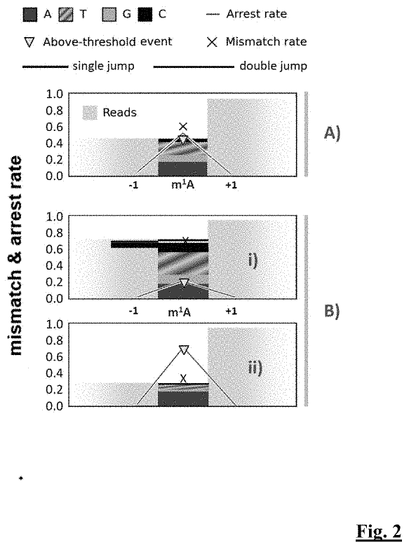

FIG. 2: A) The RT signature of a m1A site, obtained by a conventional method using a single RTase (“single RT signature”), here RTase 5 (SuperScript® III), i.e. using the RT signature of an RT approach applying only RTase 5 (SuperScript® III) according to Table 1.
B) The RT signature of a m1A site, obtained by the method according to the invention, by combining the information of the RT signature from two different RT approaches which differ in the RTase used. The RTases used were (i) RTase 12 (SuperScript® IV) and (ii) RTase 4 (GoScript™) according to Table 1.

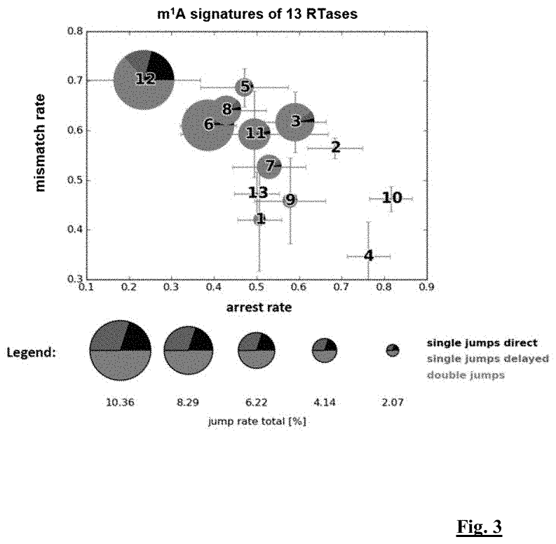

FIG. 3: m1A signatures of 13 RTases at 26 m1A sites in the cytosolic tRNA of yeast.
Error bars show standard deviations of arrest rates and mismatch rates across 3 sequencing runs, i.e. technical triplicates.
The size of pie charts represents the total jump rate, i.e. the sum of 3 types of nucleotide omission rates due to m1A sites.
Single jump direct=1 nucleotide was omitted and skipped at m1A itself.
Single jump delayed=1 nucleotide was omitted and skipped at the m1A's 5′ adjacent position.
Double jump=2 nucleotides were omitted and skipped, one at the m1A site and one at the −1 position.
The arrest rate percentages refer to the reads covering the 3′ adjacent position of m1A (+1). Mismatch and jump rate percentages refer to the reads covering the m1A position.

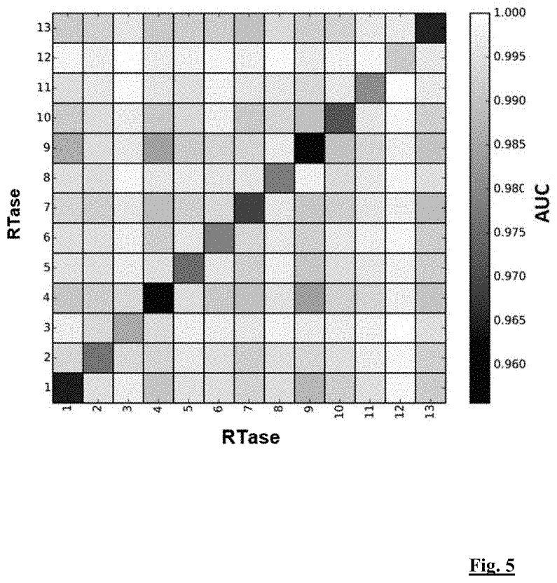

FIG. 4: Random Forest performance and weighting of RT signature features for 13 different RTases.
Classification power is represented as the Area Under Curve (AUC) of the Receiver Operating Characteristic (ROC). For each of the 13 RTases, the data of the three RT signatures determined in parallel were averaged, and the black vertical bar shows the standard deviation of the AUC.
Total Jump=total jump rate.
G, T, C=mismatch components that add up to 100%.
Weighting=mean loss in classification accuracy, if the values of the respective features are permutated, i.e. are replaced, between the training instances (m1A instances and non-m1A instances).

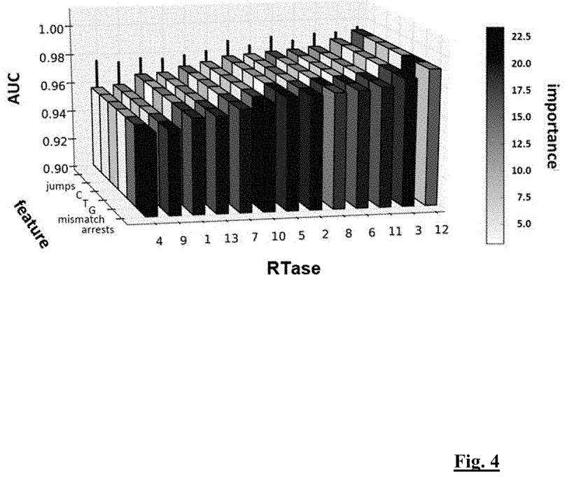

FIG. 5: Random Forest performance for determining the prediction performance using the pairwise permutationally combined RT signatures of 13 different RTases (according to Table 1), i.e. using 13×12=156 different heterogeneous RTase pairs and 13 single RTases (diagonally).
The Area Under Curve (AUC) values (bright means higher prediction performance, dark means lower prediction performance) of a receiver operating characteristic (ROC) for the RT signatures obtained from three technical replicates (i.e. from a technical triplicate) were averaged in a 100-repetitions-3-fold cross-validation of a binary classification setup with 26 positive (m1A) and 26 randomly selected negative (non m1A) cases.
Number of random forest models:

3×13×12×100×3(combined)+3×13×100×3 (not combined)=152.100

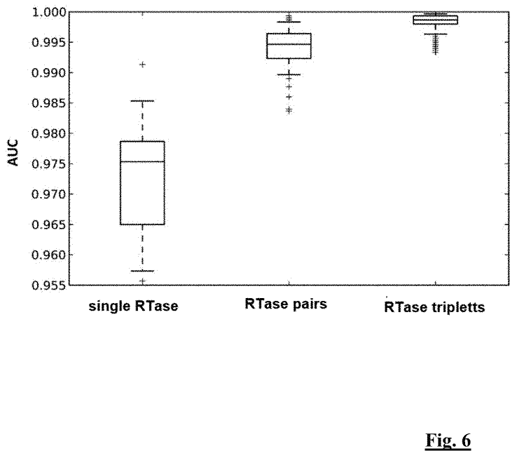

FIG. 6: box plot (box whisker plot, box graphic) for the m1A prediction performance of the Random Forest classification, which was trained with the information or data, respectively, of the RT signature features of one RT signature (of one of the 13 different RTases), of two RT signatures (of one of the 156 RTase pairs), and of three RT signatures (of one of the 1716 RTase triplets). The Area Under Curve (AUC) values of the receiver operating characteristic (ROC) from 100 repetitions of a 3-fold cross validation, applied to each of the three sequencing runs, were averaged.
The boxes indicate the area in which the inner 50% of each data population is located.
The whiskers mark the percentile values 5% and 95%, i.e. the values that form the boundary to the lower 5% and the upper 5% of the data, respectively.

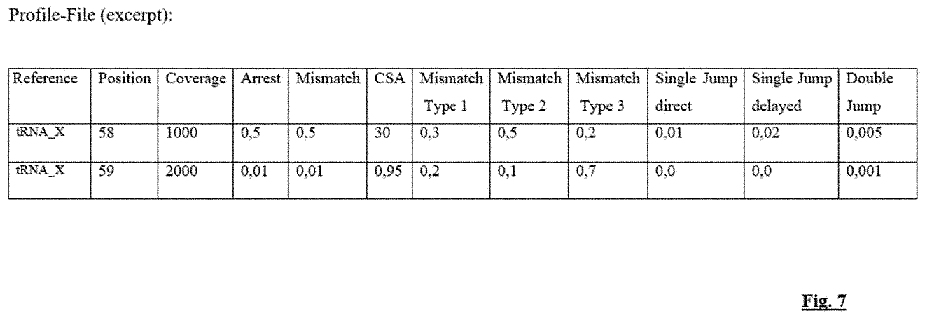

FIG. 7: An example of a Profile file.
Mismatch type 1, type 2 and type 3 are synonymous with the three concrete mismatches with the three bases that naturally occur in addition to the reference base (and its modification) in the genome; i.e. in the case of a modification of A, the mismatch types are G, T, and C.

### EXAMPLE 1

### Obtaining the Template RNAs for the Calibration Phase (Phase I)

The known RNA(s) used in the calibration phase and identified with respect to their nucleotide sequence and to the eventually existing selected nucleotide modifications were either synthetic RNAs (commercially available e.g. at IBA, Gottingen, Germany) or RNAs derived from natural sources, such as yeast RNAs whose sequence information are known from databases such as e.g. MODOMICS.

RNA Extraction from Yeast (Saccharomyces cerevisiae)

Yeast rRNA and yeast tRNA were obtained by known and common methods, e.g. as described in Tserovski et al. (2016).

0.5 μg of RNA was used per sample/batch for a reverse transcription (reaction).

### EXAMPLE 2

### Protocol for the Preparation of the cDNA Library (s)

The protocol corresponds in principle to that described in Tserovski et al. (2016).

(A) Fragmentation of Template RNA in the Case of rRNA as a Template

Total or ribosomal RNA was fragmented in a volume of 10 μl containing 10 mM ZnCl₂ and 100 mM Tris-HCl, pH 7.4, at 90° C. for 5 min. The reaction was stopped by adding ethylenediaminetetraacetic acid (EDTA) to a final concentration of 50 mM. Thereafter, the RNA fragments were size separated by 10% denaturing polyacrylamide gel electrophoresis (PAGE). 50-150 nt bands were excised from the gel, eluted in 0.3 M ammonium acetate (NH₄Ac) and precipitated with ethanol.

### (B) Dephosphorylation

The template RNA(s) (about 0.5 ii g per sample/batch) was/were dephosphorylated at both end points. The dephosphorylation mixture (total 10 μl) consisted of 100 mM Tris-HCl, pH 7.4, 20 mM MgCl₂, 0.1 mg/ml BSA, 100 mM 2-mercaptoethanol and 0.5 U FastAP Alkaline Phosphatase (Thermo Scientific, #EF0651) at 37° C. for 30 min. Before the enzyme was added, the RNA was denatured at 90° C. for 30 sec and then cooled on ice (hereinafter, this treatment is called “heat denaturation”).

After 30 minutes dephosphorylation, the RNA was heat denatured again for 30 sec and subsequently the described dephosphorylation step was performed a second time.
Total volume: 10.5 μl

### (C) 3′ Adapter Ligation

Next, an adapter was connected (ligated) to the 3′-end of the dephosphorylated RNA. The ligation (attachment) of the preadenylated 3′-RNA adapter (whose 5′ end was blocked by a C6-body) to the 3′-end of the RNA was carried out as described in Tserovski et al. (2016), using one or more ligases (in this case T4 RNA Ligase 2 truncated, New England Biolabs, #M0242L, and T4 RNA Ligase, Thermo Scientific, #EL0021) and without interposition of a purification step, in the reaction mixture of the dephosphorylation reaction with 5 μM adenylated 3′-RNA adapter, 15% DMSO, 1 U T4 RNA ligase 2 truncated and 0.5 U T4 RNA ligase. The ligation reaction was carried out at 4° C. overnight. Subsequently, the enzymes were inactivated at 75° C. for 15 min.
Total volume: 20.0 μl.

### (D) Removal of Excess Adapters

Prior to the reverse transcription step, the excess of RNA adapter was removed using the enzymes deadenylases and exonucleases (here 5′-deadenylase, New England Biolabs, #M03315 and Lambda Exonuclease, Thermo Scientific, #EN0561). For this purpose, the ligation mixture of (C) was supplemented by an amount of 20 U of 5′-deadenylase (e.g. New England Biolabs, Frankfurt, Germany) and then incubated at 30° C. for 30 min. After heat denaturation (90° C. for 30 sec., 2 min. cooling on ice), the deadenylation step was repeated with the addition of the same amount of enzyme as in the first run.
Total volume now: 22.0 μl.

Next, the digestion/degradation of the single-stranded RNA adapter (now fully monophosphorylated) was performed by adding 10 U lambda exonuclease (Thermo Scientific, Dreieich, Germany) to the reaction mixture and incubating at 37° C. for 30 min. After heat denaturation of the enzyme (90° C. for 30 sec., 2 min. cooling on ice), this digestion reaction was repeated with the addition of an equal amount of enzyme as in the first round. Subsequently, the enzyme was heat inactivated at 80° C. for 15 min.
Total volume now: 24.0 μl.

From the resulting mixture, the RNA was precipitated, here with the addition of initially 1 μl glycogen (Thermo Scientific, Dreieich, Germany, #R0561) and ammonium acetate NH₄Ac (final concentration: 0.5 M) to a total volume of 50.0 μl and subsequent addition of 150 μl ethanol per sample.

### (E) Reverse Transcription

The composition of the reverse transcription mixture was as described in Tserovski et al. (2016). The pellet obtained in (D) was first redissolved in the respective RTase-specific reaction mixture (according to the manufacturer's protocol), consisting of the reaction buffer (target final concentration 1×) and RT primer (target final concentration 5 μM), e.g. for RTase SuperScript®III=RTase 5 from Table 1 consisting of 1 μl RT primer from IBA, Gottingen, Germany, in a final concentration of 5 μM, in 4 μl First Strand (FS) buffer (e.g. from Life Technologies, Darmstadt, Germany) supplemented with water to 16 μl.

This was followed by heat denaturation at 80° C. for 10 min. with subsequent cooling on ice. Thereafter, 0.5 mM dNTP mix (=mixture containing all four deoxyribonucleotide triphosphates dATP, dGTP, dCTP and dTTP) was added and, depending on the type of RTase, in addition DTT, BSA and/or MgCl₂ (e.g. in the case of RTase SuperScript® III=RTase 5 according to Table 1: BSA, dithiothreitol), and finally 200 U of the selected RTase (z. B. 10 U/μl, SuperScript® III, Life Technologies=RTase 5 from Table 1) was added.

The transcription reactions were carried out at 45° C. for 1 hour, except for the case of using the RTase Volcano®=RTase 13 according to Table 1, where the reaction temperature was 60° C.
Total volume: 20.0 μl
   (F) Removal of Excess Primers and dNTPs

For the purpose of primer digestion/degradation, 10 U exonuclease (here: lambda exonuclease, Thermo Scientific, # EN0561) was added to the reverse transcription mixture of (E) and all incubated at 37° C. for 30 min. The reaction was repeated once by addition of an equal amount of enzyme. Heat denaturation between first and second runs was omitted to avoid denaturation of RNA:DNA-hybrids.
Total volume: 22.0 μl.

Following the second exonuclease reaction run, 40 U of single-stranded specific exonuclease I (Thermo Scientific, #EN0582) were added to the mixture and incubated at 37° C. for 30 min. Again, the reaction was repeated by addition of an equal amount of enzyme, without intermediate heat denaturation.

Finally, all enzymes were heat inactivated at 80° C. for 15 min.
Total volume: 26.0 μl

Thereafter, the dNTP residues were dephosphorylated. For this, 2 U of the heat-sensitive alkaline phosphatase FastAP (Thermo Scientific, # EF0651) were added to the mixture and incubated at 37° C. for 30 min. This was followed by heat denaturation (90° C. for 30 sec., 2 min. cooling on ice) and a repeat of the dephosphorylation step. At the end of this repeated dephosphorylation reaction, the enzyme was inactivated at 75° C. for 5 min.
Total volume: 30.0 μl

Subsequently, the degradation of the RNA was carried out by addition of NaOH (final concentration: 0.15 M), heating to 55° C. for 25 min. and then cooling on ice for 2 min. The reaction was stopped by neutralizing with an equal amount of acetic acid (final concentration: 0.15 M).

For the upcoming recovery of the cDNAs (cDNA molecules) by means of ethanol precipitation, 1 μl glycogen (Thermo Scientific, #R0561) and NH₄Ac (final concentration: 0.5 M) were added to the reaction mixture.−Total volume: 100.0 μl

Subsequently, the cDNAs were precipitated with 250 μl ethanol.

(G) 3′-Tailing and Ligation of the cDNA

For the upcoming “3′ tailing” reaction with the Terminal deoxyribonucleotidyl Transferase TdT (Thermo Scientific, #10533-065), the cDNA pellet obtained in (F) was picked up/collected and resuspended in the reaction mixture consisting of 1×TdT buffer, 1.25 mM rCTP and 1 U/μl TdT.

The mixture was incubated at 37° C. for 30 min. This was followed by a heat treatment at 70° C. for 10 min. in order to inactivate the enzyme. Total volume: 10.0 μl.

With the resulting mixture, the ligation reaction was carried out then, here e.g. by using T4 DNA ligase (Thermo Scientific, #EL0013). For the ligation of the double-stranded DNA adapter, 1.5 U/μl of T4 DNA ligase and 10 μl of ATP in 50 mM Tris-HCl at pH 7.4 and 20 mM MgCl2 were added to the mixture (final concentration of the DNA adapter: 1.25 μM), and this ligation mixture was incubated overnight at 4° C. This was followed by a heat treatment at 75° C. for 15 min. in order to inactivate the enzyme.—Total volume: 40.0 μl

The extraction of the cDNA ligation products from the mixture obtained was carried out by ethanol precipitation, initially adding 1 μl glycogen (Thermo Scientific, #R0561) and NH₄Ac (final concentration: 0.5 M) to this mixture (—total volume: 50.0 μl) and finally adding 150 μl ethanol.

Polyacrylamide gel electrophoresis (PAGE) was performed to remove excess DNA adapter. For this purpose, the pellet obtained most recently and containing the ligation products was collected and resuspended in 10 μl H₂0. This resuspended ligation product mixture was applied to a denaturing 10% polyacrylamide gel. After electrophoresis, the areas of the size range between 40 nt and 150 nt were excised from the gel and eluted overnight with 300 μM 0.5 M NH₄Ac.

The recovery of the cDNA ligation products from the eluate obtained was carried out by ethanol precipitation, initially adding 1 μl glycogen (Thermo Scientific, #R0561) (—total volume: 301.0 μl) and finally adding 750 μl ethanol.

### (H) PCR Amplification and Bar Coding

The cDNAs obtained from (G) were amplified by polymerase chain reaction (PCR) using a Taq polymerase, here e.g. the Taq polymerase of Rapidozyme (#Gen-003-1000), and P5 and P7 primers barcoded correspondingly, here e.g. with 8 nt barcodes each. For this purpose, the pellet most recently obtained in (G) with the ligation products of size 40 nt to 150 nt was collected and resuspended in 20 μl PCR reaction mixture. The PCR reaction mixture consisted, per 20 μl, of 1× Taq polymerase buffer, 3 mM MgCl₂, 5 μM P5 primer, 5 μM P7 primer, 0.5 mM dNTP mix and 0.25 U/μl Taq polymerase.

The obtained resuspension with the adapter ligated cDNAs, the P5 and P7 primers, the Taq polymerase and the dNTPs, was subjected to 12 PCR cycles. The PCR started with a denaturation step (of DNA double strands in single strands) at 95° C. for 5 min.

Subsequently, 12 cycles consisting of denaturation at 95° C. for 1 min, annealing (hybridization) at 65° C. for 1 min, and elongation at 72° C. for 1 min., were performed. The PCR was terminated with a final elongation step at 72° C. for 5 min.
Total volume: 20.0 μl.

The extraction of the PCR products, i.e. of the amplified cDNAs, was carried out by ethanol precipitation, initially adding 1 μl glycogen (Thermo Scientific, #R0561) and NH₄Ac (final concentration: 0.5 M) to this mixture (—total volume: 50.0 μl) and finally adding 150 μl ethanol.

The PCR products (amplified cDNAs) were size separated by means of 10% denaturing polyacrylamide gel electrophoresis (PAGE).

For this purpose, the pellet obtained last with the amplified cDNAs was collected and resuspended in 10 μl of H₂O. This resuspension was applied to a denaturing 10% polyacrylamide gel. After electrophoresis, the gel areas of the size range between 150 nt (the size of the adapter dimers) and 300 nt (the maximum size of PCR amplification products) were cut out from the gel and eluted overnight with 300 μM 0.5 NH₄Ac.

The recovery of the amplified cDNAs from the eluate was carried out by ethanol precipitation, initially adding 1 μl glycogen (Thermo Scientific, #R0561) (—total volume: 301.0 μl) and finally adding 750 μl ethanol.

The recovered pellet containing the amplified cDNAs of size 150-300 nt was collected and resuspended in 10 μl H₂O. The cDNAs contained in this suspension were ready for sequencing, in particular for high-throughput sequencing with NGS methods, e.g. “sequencing with bridge amplification”.

### EXAMPLE 3

### High Throughput Sequencing

### (A) Quality Control and Quantification

An aliquot of (each of) the cDNA samples obtained according to Example 2 was subjected to electrophoretic separation followed by quality control and quantification. This was preferably done by machine, here e.g. by using the Agilent Bioanalyzer 2100, an apparatus known and commonly used in the art for performing highly sensitive electrophoretic separations.

For this purpose, the aliquots were diluted (5-500 pg/μ1) and loaded on an Agilent High Sensitivity DNA chip. The thus loaded chip was introduced into the analyser. During the machine analysis, the sample components (DNA molecules) were electrophoretically separated, detected and translated into gel-like images (bands) and/or electropherograms (peaks). The data were generated in digital form and automatically analysed in real time. If the quality of the aliquot examined was satisfactory, the corresponding sample was used further.

(B) Sequencing (with NGS Methods)

The samples tested according to step (A) above and found to be of satisfactory quality were sequenced. This sequencing was carried out with the NGS method “sequencing with bridge amplification”, e.g. by using the known and common Illumina sequencing, here the MiSeq method (“MiSeq sequencing”) using the sequencer MiSeq on the MiSeq platform.

For this purpose, the samples of the cDNA librarie(s) prepared according to Example 2 (where appropriate several in parallel), which had been examined for quality and quantity and found to be suitable, were combined, denatured and diluted (10 pM) with 2 N NaOH, and applied on the support plate, the so-called “Flow Cell”.

This support plate/flow cell with the cDNA molecules of the samples was introduced into the sequencer and then sequenced mechanically (according to the manufacturer's instructions, see: MiSeq® System Manual, Catalog No. SY-41 1-9001 DOC, Material No. 20000262, Document No. 15027617 v01 DEU, September 2015).

The determined sequence information per cDNA molecule, the “reads”, were prepared and output in digital form and were ready for feeding into and further processing in a bioinformatics pipeline.

The sequencing data obtained were checked for quality and adapter contamination. For this purpose, they were examined (here and preferably) on a (the) Bioinformatics or High-throughput sequencing pipeline with the FastQC software program known in the art.

The FastQC program created a quality control (QC) report of the detected problems that had arisen either in the sequencer or in the source library material. FastQC could be run in one of two modes. It could either run as a stand-alone interactive application for instant analysis of small numbers of FastQ files, or it could run in a non-interactive mode suitable for systematically processing of a large number of FastQ files. In this non-interactive mode it was easy to integrate it into a larger analysis pipeline.

Here in the example, the examination was carried out by means of FastQC within the MiSeq RTA software. In the process step of so-called demultiplexing, first the barcode sequences from the barcoding PCR step were identified (no fault tolerance—0 mismatch) and then the reads (sequencing data) were separated into individual FastQ files (one FastQ file per sample or per original cDNA library).

These FastQ files were checked for quality, adapter dimers and over-represented sequences.

### EXAMPLE 4

### Trimming and Mapping of the Meads (Mequencing Data)

### (A) Trimming

The reads obtained according to Example 3 were trimmed, i.e. adapter sequences, in particular the sequences of the adapter P5 and P7 from the PCR reaction (see Example 3 (H)) and also random 10 nt sequences of the 3′-RNA adapter at the 3′-end of the RNA (cf. Example 2 (C)) and variable number of 5′-G RNA nucleotides from the CTP cDNA tailing step (cf. Example 3 (G)) were removed.

Trimming was done (here and preferably) computer-based with bioinformatics software for adapter trimming commonly used in the art, in this example with the Cutadapt v1.8.1 software.

### (B) Mapping

The mapping, i.e. assignment to the reference genome, was performed using the Bowtie 2 software. The settings of this Bowtie2 aligner were: alignment mode=end-to-end alignment (“global”); seed length (=length of initial alignment attempt)=6 nt−L6; k=1 (i.e. when mapping of all references simultaneously, only one alignment declared as valid by Bowtie2 was reported for each read), and mismatch=−N1 (i.e. tolerance of one mismatch in the “seed”, i.e. in the area of the initial alignment attempt).

### EXAMPLE 5

### Diagnosis of the RT Signature

RT signature diagnostics, i.e. the identification and quantitative measurement of the reverse transcription event pattern for the subject template RNA(s) was done at each individual nucleotide position of the RNA of interest using software programs known and commonly used in the art, e.g. the SAMtools software (version 1.2).

For this purpose, first the SAM files from the mapping step were converted into BAM files. Then the following steps carried out: (i) sorting and indexing the BAM files, (ii) converting the BAM files to the Pileup format, and (iii) converting the Pileup format into a user-defined tab-delimited text file (so-called “Profile File”).

For each nucleotide position (reference position) of the template RNA(s), the files displayed all relevant RT signature features, such as coverage, arrest rate, total mismatch rate, single mismatch rates (of the respective mismatched nucleotides), total jump rate, single jump rate direct, single jump rate delayed, double jump rate.

Features were collected based on the Pileup format and calculated in Profile format according to the following rules:

The arrest rate aᵢ of a position i is defined as the relative proportion of reads (cDNAs) starting at the location i+1, i.e. covering i+l but not i, among all reads that cover i+1 and whose number is referred to as coverage cᵢ₊₁. If sᵢ₊₁ is the number of reads starting at i+1, then the arrest rate of position i is defined as aᵢ=sᵢ₊₁/cᵢ₊₁. Let be dᵢ the number of mapped reads covering i with a base that differs from the reference base i. Then the mismatch rate is defined as mᵢ=dᵢ/cᵢ. The single mismatch rates for G, T and C at a m1A site are counted as fractions of mᵢ. The mentioned numbers of coverages, arrests, starts, mismatches and jumps can be determined directly from the pileup format. Each line of the pileup format accurately reflects the coverage of a reference position. Points and commas represent overlapping bases that resemble the reference base. Bases that differ from the reference base (in the template RNA) appear in Pileup format in the form of the usual letters A, G, T or C (if the respective read has been aligned in “sense” direction, i.e. as it is) or a, g, t, or c (if the respective read has been aligned in “antisense” direction, i.e. as its reverse complement). A jump over the corresponding position is displayed as an asterisk. In the case of jumps over multiple positions, a minus sign is placed at the first position instead of the asterisk, followed by a number that represents the number of skipped positions. For calculating the so-called context-sensitive arrest rate CSA (CSA is defined as the ratio of position-specific RT arrest aᵢ of a position i to the RT arrest observed in the local environment, i.e. in the adjacent sequences) in Pileup format 5 digits before and 5 digits after the respective nucleotide position i (i.e. the neighbouring sequences five bases upstream (+5 bp) and five bases downstream (−5 bp)) are used, and the arrest rate at position i is divided by the median of the arrest rates of all eleven positions in this window. The window size may be increased or decreased depending on the knowledge of the nature of the present RNA, to improve, if appropriate, the prediction performance in the cross-validation.

The data thus obtained were transformed from the Pileup format into e.g. (as here and preferably) the Profile format and stored there and displayed as needed. FIG. 7 shows an example of such a Profile file.

For further processing of the data e.g. in a classifying procedure intended to classify only nucleotide positions of a particular reference base (A, C, G or T), e.g. of adenine (A) to either “modified” (m1A) or “unmodified” (A), Profile files with reduced data set may be created, e.g. only with the data of positions corresponding to the reference base A of the considered modification m1A.

### EXAMPLE 6

### Computer-Based and Machine-Learning-Based Supervised Prediction of M1A Sites

The digital data of the RT event pattern (the RT signature) obtained according to example 5, which preferably were available in the form of Profile files, were fed into a computer-based and machine learning-based classification system, e.g. (here and preferably) in the Random Forest classification system (R Version v3.3.1) based on decision trees and known and common in the art.

For classifying the RT signatures, the classifier was fed with at least the attributes: arrest rate a, total mismatch (mismatch) rate m, the m/a ratio, relative mismatch composition (fraction content of G, T and C), and the context-sensitive arrest rate CSA, and preferably also the jump rate.

### (A) Training and Testing (Review) of the Classifier (Phase I of the Method)

For training and testing/checking the classifier for the detection of a particular characteristic RT signature, here e.g. for the detection of the RT signature at m1A sites, the algorithm was initially fed with equal numbers (e.g. 45 each as described in Hauenschild et al. (2015)) of RT signatures of known m1A sites (from known template RNAs with identified m1A sites) and RT signatures of known unmodified (or not identifiable modified) A sites (from known template RNAs without m1A sites).

For this purpose, Profile files with a reduced data set created according to example 5 were preferably used, i.e. Profile files which only contain the RT signature data of the positions displayed for the reference base in question (here in the example m1A or non-m1A positions for base A). Preferably, m1A-similar non-m1A sites were included in the training in order to prepare the classifiers for the detection or prediction of difficult cases in unknown template RNAs (as described in Hauenschild et al., 2015).

Using the RT signatures of the known positive m1A sites the classifiers (here, for example, and preferably the Random Forest classifier, R version v3.3.1) created (“learned”) and adapted (corrected and optimized) the particular and typical (characteristic) profile for the m1A site (cf. FIG. 2 A). In other words: the classifier implicitly learned the typical m1A RT signature profile during the training and (self-) testing/verification runs.

As an optional quality check, a repeated, multiple (here e.g. triple) cross validation was performed. However, this measure can also be omitted.

The classification result consisted of a listing of all checked positions (“instances”) on the template RNA(s), with a rating per position (“instance”) with regard to the decision or question as to whether the respective RT signature in question corresponded approximately or completely with the learned typical m1A RT signature profile (cf. FIG. 2A), i.e. was similar to it or matched it.

The evaluation was carried out by specifying a numerical value between 0 and 1, wherein the value “0” corresponds to a clear “no” and the value “1” to a clear “yes”. Intermediate values represent a corresponding probability for a “yes” or a “no” (e.g. value 0.99 represents a relatively very certain “yes” and value 0.4 represents a weak “no”). The closer a score is to the value 1, i.e. the more certain the “yes” rating is, the stronger it weighs as an indicator of the presence of the m1A nucleotide modification at the relevant position of the template RNA.

The mean predictive performance (sensitivity, specificity) was calculated on the basis of training and testing/verification using cross-validation.

### (B) Use (Application) of the Classifier for Studying an Unknown Template RNA

The unknown template RNA(s) to be examined (for example from a patient sample) was prepared according to Examples 2 and 3. The sequence information obtained in the form of reads were trimmed according to Example 4 and mapped to a reference genome, and examined according to Example 5, i.e. the RT signature features coverage, arrest rate, total mismatch rate, single mismatch rates (of the relevant single mismatched nucleotides), single jump rate direct, single-step rate delayed, double jump rate were tested for presence at each nucleotide position and, if necessary, measured quantitatively.

Preferably, and for the purpose of minimizing the expenditure, according to example 5 only those nucleotide positions which have the reference base in question (the nucleoside in question) were stored in the Profile format and fed into the classifier.

In the case of application for investigating possibly existing m1A sites, consequently all potential positions with the reference base A (adenine) were stored in Profile format and fed into the classifier, here the Random Forest.

A shortened runtime of the prediction procedure could be achieved by removing obviously unsigned lines (i.e. nucleotide positions that were correctly transcribed by RTase, and thus, where none of the characteristic RT signature features are present) from the Profile file in a controlled manner with the help of a simple filter (requesting user-selected minimum values for signature features).

As a result of the investigation, the classifier (e.g. the Random Forest model) provided an assessment between “yes” and “no” for each nucleotide position in the examined transcriptome, and thus provided a list of those positions on the unknown template RNA(s), which had an RT signature that approximately or completely matched/corresponded to the specific and typical (characteristic) profile (implicitly learned by the classifier) for the nucleotide modification site in question, here in the example the m1A site.

Here (for example and preferably), in each case the so-called “score”, a numerical score on a one-dimensional rating scale, was additionally indicated, as a measure of the quality of the rating. The creation of the score is a (possible) component of the Random Forest classifier.

### EXAMPLE 7

### Analysis and Comparison of the RT Cignatures of 13 Different RTases for the (or at) RNA Nucleotide Modification m1A

Analogously to the method described in Examples 2 to 5, the 13 different reverse transcriptases (RTases) known in the art and commercially available were examined in parallel and analogously with respect to their respective RT signature at m1A sites.

Generation of the cDNA libraries and high-throughput sequencing (according to Examples 2 and 3) as well as trimming and mapping and diagnosing of the RT signatures (according to Examples 4 and 5) was repeated three times for all 13 RTases (i.e. triplicates were generated for each RTase). As template RNA, the well-analysed (annotated) total tRNA of Saccharomyces cerevisiae (available e.g. from Roche Diagnostics: ref 10109525001/lot 13407921), which contains a sufficient number of known m1A sites, was used for each of the RTases. The reference sequences used were a set of 43 tRNAs, compiled from the databases MODOMICS (Machnicka et al., 2013) and Sprinzl (Rifling et al., 2009). Among these 43 tRNAs there were 26 tRNAs bearing an m1A in their sequence.

The detected RT signatures of the 13 different RTases showed large variations in their arrest and mismatch rates, i.e. the arrest and mismatch rates of the individual RTases were very different in comparison with each other (see FIG. 3). For example, RTase 10 (MonsterScript™) and RTase 4 (GoScript™) showed high arrest and low mismatch rates, whereas RTase 12 (SuperScript® IV) showed an inverse behaviour in comparison.

These differences indicate that the detection capacity of the individual RTases greatly varies with respect to m1A positions in the sequence of the template RNA.

In the course of these comparative studies, a hitherto unknown phenomenon was surprisingly found, which was of varying severity in the individual RTases: in the sequencing data sets of some RTases, characteristic sequence gaps were recognizable at m1A sites, which indicate jumps in the transcription of the RNA into cDNA. In other words, in addition to mismatch and arrest, in their RT signature the RTases in question also showed characteristic gaps in the sequence reading, a previously unknown phenomenon, resulting from jumps of the RTase concerned across the m1A position (see FIG. 1 and FIG. 2).

These jumps were noticeably frequent at m1A sites, and in particular at those with a high coverage due to the strong read-through capability of the respective RTase.

Single and double jumps can be distinguished. The single jumps may be direct or delayed single jumps, that is, the (skipped) gap occur either directly at the m1A site or at the location of its 5′ adjacent neighbour, known as the −1 position. Double jumps lead to and appear as gaps at the two positions m1A- and −1.

The variability of the jump capabilities, ranging from a total jump rate of about 10% for SuperScript® IV to negligible values for RTases with highest arrest rates, represents another level of RTase's individual read-through capability. Generally, most of the occurring jumps are double jumps over two nucleotides. Single jumps occur approximately equally frequently, with a slight preference for delayed single jumps.

The scatter plot in FIG. 3 shows the great variety of RT signatures at m1A sites for the 13 different RTases examined, taking into account this newly discovered third core feature “jumps”. (The given values of arrest rate, mismatch rate, and total jump rate, each represent the average of the three individual values concerned of each technical triplicate, and the error bars show the standard deviations of the arrest and misincorporation rates of these triplicates.)

The use of the observed large variance in the RT signature of the 13 different RTases (see FIG. 3)—taking into account only the characteristic features arrest rate and mismatch rate or all three features recognized as defining, i.e. arrest rate, mismatch rate and total jump rate—, offers greatly improved and extended possibilities for the detection of m1A sites—or of other nucleotide modification sites with typical RT signatures—in any test template RNA.

### EXAMPLE 8

### Evaluation of the Prediction Performance of the RT Signature of an RTase and Weighting (Significance) of the RT Signature Features

To evaluate the prediction performance of the RT signature of an individual RTase, for the RT signatures obtained in Example (7) of the 13 RTases at m1A locations, it was determined in each case in accordance with Example 6 (A), which of the characteristic features of the RT signature with which weighting defines or co-defines the relevant RTase-specific RT signature. The following six RT signature characteristics were examined (here for example, and preferably): arrest rate, overall jump rate and mismatch rate, and with regard to mismatch events, also the relative contents of the mismatch components G, T, and C.

In this study also, large differences between individual RTases were found (cf. FIG. 4). For some RTases, arrest rate and mismatch rates dominated their RT signature, and therefore their predictive power is mainly based on arrest rate and mismatch rate, while in other RTases, the jump rate strongly influenced the RT signature and should therefore be included in the evaluation of RT signature prediction performance.

To determine how the RTase-type specific differences in RT signatures affect the discrimination (demarcation) between m1A and non-m1A instances, a supervised machine learning experiment was performed:

For each of the 13 RTases listed in Table 1, the RT signatures of 26 m1A instances (obtained with m1A-containing yeast tRNAs as template RNAs) were paired with an equal number of non-m1A signatures, randomly drawn previously from the surrounding sequence pool. These pairs were shuffled and divided into three groups of equal class frequency (so-called “folds”). Each signature data point contained the RT signature features arrest rate, relative mismatch rate, relative mismatch components (G, T and C), and the total jump rate. In a cross validation, a random forest model (as described in Liaw and Wiener, 2002) was trained on two of these groups (“folds”) and tested on the third one. Shuffling (i.e. mixture of group composition) and cross-validation were repeated 100 times, in order to account for statistical variance.

The application of this procedure to the RT signatures of each RTase obtained in Example 7 provided the results shown in FIG. 4: for each of the six RT signature features of each of the 13 RTases (No. 1 to No. 13), the averaged ranking of their performance for a m1A prediction and thus their “classification power” is specified as (in the form of) “Area Under Curve (AUC)” of the “Receiver Operating Characteristic (ROC)”. The data of the RT signature triplicates were averaged; the black vertical lines show standard deviations over 3 sequence runs). For each RTase, 100 repetitions of a 3-fold cross-validation were performed in each classification run. Each binary classification setup contained 26 positive (m1A) and 26 randomly selected negative instances (non-m1A, i.e. without nucleotide exchange) from the tRNA sequence space and was split under stratification into two training and one testing data group. This leads to 3×13×100×3=1 1,700 Random Forest models.

It is clearly recognizable that RT signatures of some RTases led to better prediction performance results than those of other RTases. For some RTases, the prediction performance results differed by several percentage points. The RTase 5 (SuperScript® III) used in the investigations described by Hauenschild et al. (2015) ranked in the middle of this classification. Through targeted choice of the presumptive most suitable RTase(s) for a planned sequence examination of a test template RNA for a given nucleotide modification, the workflow can be improved and residual errors can be significantly reduced.

A comparison of the weighting values, obtained for the RT signature features of different RTases (at m1A sites) with machine learning models designed for that purpose, indicates an individual weighting pattern of the features in the RT signature of each RTase that contributes to the decision making.

The determination of the weighting (synonyms: significance, importance) of an RT signature feature (e.g. arrest rate) was performed by permuting the (all) values obtained for this feature with all 13 RTases examined (i.e. transposition of values, including negative ones, namely nucleotide sites with potentially weak expression of this feature, with corresponding values of positive instances, namely nucleotide sites with mostly stronger expression of this feature), and measurement of the corresponding decrease in the classification accuracy. This decrease in classification accuracy tends to be the higher, the more important this feature is for the RT signature. For example, for the RT signature of RTase 3 (ProtoScript® II), the permutation of the pronounced arrest rate has major consequences, while this feature is of subordinate importance for RTase 12 (SuperScript® IV), where it is expressed only marginally. Although they differ in feature weighting patterns, RTase 3 (ProtoScript® II) and RTase 12 (SuperScript® IV) occupy the highest AUC ranks, i.e. their RT signatures have the strongest classifying ability and allow the best machine learning performance.

### EXAMPLE 9

### Comparison of m1A Detection Performance—Pairwise Combination of RT Signatures of Different RTase Types (a) after Single Performances and (b) with Different Patterns in the Weighting (Importance, Importance) of their RT Signature Features

To test whether these differences in the RT signatures of different RTases, observed according to Example 8, can be used for improved detection of nucleotide modification sites in template RNAs, the supervised machine learning experiment described in Example 8, namely the Random Forest training and testing/verification (based on 100 repetitions of a 3-fold cross validation) was carried out for RTase pairs, i.e. using the RT signature data (cf. FIG. 3) of two RT-signatures of two different RTases each. Thus, for the total of 13 different RTases (according to Table 1), there were 13×12=156 heterogeneous RTase combinations, i.e. pairs of two different RTases. For comparison, this learning experiment was also carried out analogously with the unpaired RT signatures of the 13 individual (single) RTases. Instead of six learning features according to example 8 (arrest rate, total jump rate, mismatch rate and relative content of the mismatch components G, T and C) twelve learning features (namely twice these six) were now specified for each training session. The evaluation of the 156 RT signature data combinations showed a measurable improvement in the (m1A) prediction performance (AUC values) for each of the pair combinations of two different RTases and a particularly significant improvement for RTases with pronounced differences in the weighting of their RT signature features (see heatmap in FIG. 5). For example, the combination of two powerful RTases, such as RTase 12 (SuperScript® IV) and RTase 3 (ProtoScript® II), provided highest AUC values and thus the best prediction performance. In contrast, the individual/single (unpaired) RTases (cf. FIG. 5: diagonal field series) delivered significantly lower AUC values and thus poorer prediction performance. These results clearly demonstrate that the combined use of two RT signatures derived from two different RTases provides much better prediction than the use of simple RT signatures of a given RTase. This significant improvement or performance enhancement indicates that it relies on a synergistic effect of the combination of RT signatures of different RTases.

Consequently, with the help of the correct RTase combination, a significantly optimized prediction performance for the RT signatures obtained (of the RTase pairs) can be achieved. By targeted selection of two (or more) RTases with known RT signatures at the nucleotide modification site concerned, the combination of which is probably best suited for a planned project when taking into account the weighting of their RT signature characteristics, and by the use of these RTases in parallel reverse transcription reactions with the same template RNA, two (or correspondingly more) RT signatures will be obtained whose combined use in the supervised machine learning experiment according to Example 8 causes (results in) a significantly improved classification performance, i.e. performance for m1A prediction. Residual errors are significantly reduced.

### EXAMPLE 10

### Comparison of m1A Detection Performance—Use of RT Signature Triplets for Performance Enhancement

The studies described in Example 9 were carried out analogously with RTase triplets, i.e. with triple combinations of different RTases.

As expected, the results obtained demonstrated that the prediction performance (detection performance) for a m1A site can be improved by combining the RT signature data from three different RTases. Numerous RTase triplets yielded AUC values of 1,000 thereby demonstrating an ideal classification performance (“classification power”) and m1A prediction performance, respectively. However, a detailed comparison between RT signature pairs and RT signature triplets reveals that the RT signatures of some RTase pairs already provide a quasi-best m1A prediction performance, i.e. their AUC values are in a range that overlaps with that of the RT signature triplets.

FIG. 6 shows a graphical comparison by using a box plot of the m1A prediction performance in a Random Forest classification for the three alternative training and application modes (states) of the classification method, namely training (according to Example 6 A) and application (according to Example 6 B) with the information or data of the RT signature features of (i) an RT signature (of the 13 different RTases), (ii) two RT signatures (of one of the 156 RTase pairs) and (iii) three RT signatures (of one of the 1716 RTase triplets).

These results, according to which the prediction of a nucleotide modification site m1A with the highest possible probability of hit (AUC equal to or approximately equal to 1,000) by using only two different RTases or their RT signatures, respectively, is sufficient, indicate that even the use of two (or more) different RT signatures from parallel RT reactions (reaction runs) with the same RTase but with different reaction conditions per batch (e.g. different concentrations of dNTPs, different divalent cations such as Mg2+ or Mn2+, different concentrations of divalent cations, different pH, different temperatures, different concentrations of polyethylene glycol) allows an almost optimal m1A prediction performance (and thus a near-detection performance).

### EXAMPLE 11

### Detection of Other Nucleotide Modifications (as m1A) e.g. m1G or m2,2G

The method for detecting nucleotide modifications other than m1A, e.g. of m1G or m2,2G, is carried out as described in examples 1 to 9 or 1 to 10, with the modification that in step 1 of phase I as template RNAs and thus as training RNA set for the classifier such RNA sequences are used, which are known and proven to contain the sought-after nucleotide modification, i.e. m1G or m2,2G.

### EXAMPLE 12

### Kit for Carrying Out the Process According to the Invention

The kit comprises (a) at least two reverse transcriptases RTase X and RTase Y, whose RT signatures at the respective/relevant nucleotide modification site with respect to the weighting of the RT signature features (termination rate, total mismatch rate, single mismatch rates of the respective mismatched nucleotides, total hopping rate, direct single hopping rate, delayed single hopping rate, double hopping rate) have a different pattern in at least one of the RT signature features, and/or (b) at least two different premixed reaction batches (synonyms: reaction mixtures; buffer mixtures) A and B, which embody different (i.e. divergent) reaction conditions by e.g. containing different concentrations of dNTPs, and/or different divalent cations, in particular Mg2+ and Mn2+, and/or different concentrations of divalent cations and/or different pH values, and/or different concentrations of polyethylene glycol (PEG).

To carry out step (1) in phase (I) and in phase (II) of the process, it is only necessary to mix and incubate the RTase(s) with the reaction batch or the reaction batches and the relevant template RNA(s).

Examples of parallel reaction batches for carrying out the method according to the invention are:
(i) Batch a: template RNA (s)+RTase X+reaction mixture A
   Batch b: template RNA (s)+RTase Y+reaction mixture A
(ii) Batch a: template RNA (s)+RTase X+reaction mixture A
   Batch b: template RNA (s)+RTase Y+reaction mixture A
   Batch c: template RNA (s)+RTase X+reaction mixture B
   Batch d: template RNA (s)+RTase Y+reaction mixture B
(iii) Batch a: template RNA (s)+RTase X+reaction mixture A
   Batch b: template RNA (s)+RTase X+reaction mixture B
   Batch c: template RNA (s)+RTase X+reaction mixture C.

### REFERENCES CITED

Hauenschild, R., Tserovski, L., Schmid, K., Thüring, K., Winz, M. L., Sharma, S., Entian, K. D., Wacheul, L., Lafontaine, D. L. J., Anderson, J., Alfonzo, J., Hildebrandt, A., Jaschke, A., Motorin Y., Helm, M., “The reverse transcription signature of N-1-methyladenosine in RNA-Seq is sequence dependent”, Nucleic Acids Research, vol. 43, no. 20, pp. 9950-9964, 2015
Dominissini, D., Nachtergaele, S., Moshitch-Moshkovitz, S., Peer, E., Kol, N., Ben-Haim, M. S., Dai, Q., Di Segni, A. et al., “The dynamic N1-methyladenosine methylome in eukaryotic messenger RNA”, Nature, vol. 530, no. 7591, pp. 441-446, 2016
Linder, B., Grozhik, A. V., Olarerin-George, A. O., Meydan, C., Mason, C. E., Jaffrey, S. R., “Single-nucleotide-resolution mapping of m6A and m6Am throughout the transcriptome”, Nature Methods, vol. 12, no. 8, pp. 767-774, 2015
Liaw, A., Wiener, M. “Classification and regression “Classification and regression by randomForest”, R News, vol. 2, pp. 18-22, 2002
Tserovski, L., Marchand V., Hauenschild R., Blanloeil-Oillo F., Helm M. and Motorin Y., “High-throughput sequencing for 1-methyladenosine (m′A) mapping in RNA”, Methods, 107, 110-121, 2016
Machnicka, M. A., Milanowska, K., Osman Oglou, O., Purta, E., Kurkowska, M., Olchowik, A., Januszewski, W., Kalinowski, S., Dunin-Horkawicz, S., Rother, K. M. et al. (2013) MODOMICS: a database of RNA modification pathways-2013 update. Nucleic Acids Res., 41, D262-D267.
Jühling, F., Mörl, M., Hartmann, R. K., Sprinzl, M., Stadler, P. F. and Pütz, J. (2009) tRNAdb 2009: compilation of tRNA sequences and tRNA genes. Nucleic Acids Res., 37, D159-D162.

TABLE 1
Reverse Transcriptases

| | Reverse Transcriptase | Supplier |
|---|---|---|
| 1 | M-MuLV | New England Biolabs ® |
| 2 | AMV | New England Biolabs ® |
| 3 | ProtoScript ® II | New England Biolabs ® |
| 4 | GoScript ™ | Promega |
| 5 | SuperScript ® III | ThermoFisher ® |
| 6 | RevertAid ® | ThermoFisher ® |
| 7 | AccuScript ® | Agilent Technologies |
| 8 | AffinityScript ® | Agilent Technologies |
| 9 | M-MuLV | Promega |
| 10 | MonsterScript ™ | Epicentre ® |
| 11 | EpiScript ™ | Epicentre ® |
| 12 | SuperScript ® IV | ThermoFisher ® |
| 13 | Volcano ® | myPOLS Biotec GmbH |

1. A method for determining number and position (locus) of a selected (predetermined) known nucleotide modification in one RNA or multiple RNAs (incl. Transcriptome), the template RNA(s), comprising the following steps in the specified order:
(1) Reverse transcription of the template RNA(s) using the enzyme reverse transcriptase and creating a cDNA library containing the reverse transcription products (=cDNAs) of the reverse transcriptase used with this/these template RNA(s),
(2) Amplifying the cDNAs and sequencing the amplified cDNAs using a high-throughput sequencing method (next generation sequencing (NGS) method), the recovered sequence data being output in digital form, i.e. in the form of reads,
(3) Adapter trimming (=removal of the adapter sequences) and mapping (=assignment) of the sequenced cDNAs/reads to the reference genome or reference transcriptome by means of computerized alignment methods,
(4) computerized evaluation (analysis) of the mapping result with respect to the reverse transcription event pattern, the RT signature, using the events ‘arrest’ and/or ‘read-through with mismatch’ as RT signature feature(s), and diagnosing the RT signature at each nucleotide position of the template RNA(s),
(5) Feeding the digitized data of the RT signatures into a computerized, automated, machine-learning based classification system,
wherein in a first phase (I) of the method, the calibration phase,
steps (1) to (5) are carried out with one or several different RNAs as template RNAs, this/these RNA(s) are known and identified and annotated with respect to nucleotide sequence and optionally present nucleotide modification(s),
and RT signatures, determined in step (5), of nucleotide positions with the known nucleotide modification and of nucleotide positions of the same nucleoside without nucleotide modification, are fed into the classification system,
and the classification system during training and self-testing (classification) runs implicitly creates and optimizes (“learns”) the (characteristic) profile of the RT signature (i.e. the characteristic quantitative expression of the RT signature features) at the nucleotide position having the nucleotide modification, and (consequently) as a classification result, it determines and indicates those positions on the (each) template RNA(s) which have an RT signature that approximately or fully matches this (characteristic) profile and thus indicates the presence of the relevant nucleotide modification at these positions,
and wherein in a second phase (II) of the method, the application or examination phase,
steps (1) to (5) are carried out with one or more unknown test RNA(s) to be examined as template RNA (s),
and steps (1) to (4) are carried out under the same conditions as in Phase (I),
and RT signatures determined in step (5) of nucleotide positions of the test template RNA(s) are fed into the classification system,
and based on the (characteristic) profile implicitly learned in phase (I) step (5) the classification system classifies the entered RT signatures with regard to the criterion to what extent they are similar to or match this profile, and wherein classification results with the statement “similar” or “approximately matching” or “matching” indicate the presence of the subject nucleotide modification in the test template RNA(s) at the nucleotide position with this RT signature, and wherein in step (1) of phase (I) and phase (II) of the method, the reverse transcription of the template RNAs is carried out in two or more reaction mixtures and reaction runs with different reverse transcriptases under the same reaction conditions and/or with the same reverse transcriptase(s) under different reaction conditions per batch, wherein a cDNA library is obtained with/from each batch,
and wherein in step (4) of phase (I) and phase (II) of the method, the evaluation of the mapping results with regard to the RT signature is carried out by using the events ‘arrest’ and/or ‘read-through with mismatch’ and/or the additional event ‘read-through with sequence gap(s)’ as RT signature feature(s),
and wherein in step (5) of phase (I) and phase (II) of the method, data of RT signatures, from the cDNA libraries obtained in step (1) with the different reverse transcriptases under the same reaction conditions and/or with the same reverse transcriptase(s) under different reaction conditions, are fed into the classification system.

2. The method according to claim 1, wherein in step (1) of phase (I) and phase (II) of the method, the analogous reaction mixtures and reaction runs are carried out with at least two reverse transcriptases, whose RT signatures at or for the nucleotide modification site in question have a different pattern with regard to the weighting of their RT signature features.

3. The method according to claim 1, wherein the different reverse transcriptases used in step (1) of phase I and phase II comprise reverse transcriptases which were modified for this purpose by mutations.

4. The method according to claim 1, wherein the different reaction conditions are different concentrations of dNTPs, and/or different divalent cations, in particular Mg2+ and Mn2+, and/or different concentrations of divalent cations and/or different pH values and/or different temperatures and/or different concentrations of polyethylene glycol (PEG).

5. The method according to claim 1, wherein the nucleotide modification is a nucleoside methylation, in particular a N1-methylation of adenosine or guanosine.

6. The method according to claim 1, wherein in step (2) of phase (I) and phase (II) the sequencing is a sequencing with bridge amplification, in particular an illumina sequencing method.

7. The method according to claim 1, wherein in step (3) of phase (I) and phase (II) the alignment method is a method for sequence alignment and sequence analysis, in particular a method according to Bowtie 2 software.

8. The method according to claim 1, wherein the classification system in step (5) of phase (I) and phase (II) is a Random Forest classifier.

9. The method according to claim 1, wherein sequence data obtained in step (2) of phase (I) and (II) for carrying out steps (3) to (5) of phase (I) and (II) are fed into a bioinformatics pipeline which controls the combination of steps (3) to (5).

10. The method according to claim 1, wherein the known RNAs in phase (I) step (1) are synthetic RNAs or natural RNAs isolated according to database information.

11. The method according to claim 1, wherein for each classification result in phase II step (5) a numerical score is given on a one-dimensional numerical rating scale as a measure of the quality of the match.

12. The method according to claim 1, wherein in step (4) of phase (I) and phase (II) of the method, for the evaluation of the mapping results with respect to the RT signature, the events ‘arrest’ and ‘read-through with mismatch’ and ‘read-through with sequence gap(s) (jump)’ are determined and evaluated as RT signature characteristics.

13. A kit for carrying out the method according to claim 1, wherein the kit comprises at least two reverse transcriptases (“RTases”) whose RT signatures at the relevant nucleotide modification site, with respect to the weighting of the RT signature features, have a different pattern in at least one of the RT signature features, and/or that it comprises at least two different premixed reaction batches which preferably contain different concentrations of dNTPs, and/or different divalent cations, in particular Mg2+ and Mn2+, and/or different concentrations of divalent cations, and/or different pH-values, and/or different concentrations of polyethylene glycol (PEG).
